(19) United States
(12) Patent Application Publication
(10) Pub. No.: US 2017/0303978 A1
(43) Pub. Date: Oct. 26, 2017

(54) RIGID FIXATION SYSTEMS FOR CARDIOTHORACIC FIXATION

(71) Applicant: Arthrex, Inc., Naples, FL (US)

(72) Inventors: Matthew Palmer, Medford, MA (US); Matthew Fonte, Concord, MA (US); Robert Devaney, Auburndale, MA (US)

(21) Appl. No.: 15/481,585

(22) Filed: Apr. 7, 2017

Related U.S. Application Data
(60) Provisional application No. 62/325,317, filed on Apr. 20, 2016.

(51) Int. Cl. A61B 17/80; A61B 17/00; A61B 17/064

(52) U.S. Cl. CPC A61B 17/8004; A61B 17/8085; A61B 17/8076; A61B 17/0642; A61B 2017/00862

(57) ABSTRACT

A rigid fixation system and methods of treatment are disclosed herein to facilitate healing of diseased or damaged tissue by maintaining compression between bone fragments.

### CROSS REFERENCE TO RELATED APPLICATIONS

The application claims priority to U.S. Provisional Application No. 62/325,317 which was filed on Apr. 20, 2016.

### FIELD OF THE INVENTION

The present invention relates to a rigid fixation system for cardiothoracic fixation in a human or animal body to facilitate healing of diseased or damaged tissue by maintaining compression between bone fragments. While the invention has application throughout the body, its utility will be illustrated herein in the context of the repair of fractured or displaced bone tissue and specifically the repair of the sternum following median sternotomy and the repair of ribs after fracture.

### BACKGROUND OF THE INVENTION

In the field of cardiothoracic surgery, it is common to rejoin broken bones. The success of a surgical procedure often depends on the ability to reapproximate the fractured bones, the amount of compression achieved between the bone fragments, and the ability to sustain that compression over time. If the surgeon is unable to bring the bone fragments into close contact, a gap will exist between the bone fragments, and bone tissue will need to fill the gap before complete healing can occur. Furthermore, gaps between bone fragments that are too large can allow movement between the bone fragments, disrupting the healing tissue and therefore slowing the healing process. Optimal healing requires that the bone fragments be in close contact with each other and for a compressive load to be applied and maintained between the bone fragments. Compressive strain between the bone fragments has been found to accelerate the healing process in accordance with Wolf's Law.

In the field of cardiothoracic surgery, it is common to rejoin broken bones with wire. The wire may be passed transversely through the sternum in either a simple uninterrupted or figure-of-eight fashion. The wire may be passed either peristernal or transsternal. Twisting of the wire ends generates tension in the wire and compresses the broken bones together.

While wire fixation is designed to bring the bone fragments into close contact and to generate a compressive load between the bone fragments, wire does not always succeed in accomplishing this objective. Inter alia, closure of a median sternotomy using wiring is a tedious and time-consuming method. Three percent of patients will develop a sternal nonunion, and patients complain of pain and clicking due to the lack of rigid fixation. Wire cerclage has been shown to result in significant movement and sternal separation and does not provide adequate stability to support bone growth and healing. The lateral tension created by chest wall movement during physiological respiration parallels an axis of the wire and can cut or fracture the bone.

Thus, there exists a clinical need for a rigid fixation device which can generate and maintain compression between the fractured bones.

### SUMMARY OF THE INVENTION

A novel rigid fixation device for cardiothoracic fracture fixation is provided.

Among other things, a compression plate or staple is manufactured from a shape memory material (e.g., a material capable of exhibiting superelasticity and/or a temperature-induced shape change). The shape memory material may be a metal alloy (e.g., Nitinol) or a polymer (e.g., appropriately processed polyether ether ketone, or PEEK). The compression plate or staple is designed to generate and maintain compression between bone fragments.

The device applies the principles of tension banding to a rigid fixation system. Tension banding is traditionally accomplished using a combination of wires and pins. A tension band converts tensile forces into compressive forces on an opposite cortex by providing a buttress. When bones are eccentrically loaded, one side of the bone is under tension, while the opposite side in under compression. If a fracture is to unite, mechanical stability is required, which is obtained by compression of the fracture site. Tensile forces interfere with fracture healing and need to be neutralized or converted to compressive forces. The plate remains under tension while the bone is compressed.

Following a sternotomy, an anterior cortex will experience tensile forces (pulling the bones apart), and an opposite cortex will experience compressive forces (pushing the bones together).

In one example, the rigid fixation device is a compression plate. The compression plate can be placed on a proximal cortex and secured to a bone with cancellous bone screws. The screw can thread into holes on the compression plate to lock the two together as a rigid structure. The compression plate converts tensile forces on a proximal cortical surface to compressive forces on an opposite cortical surface.

The compression plate includes two opposing regions joined together by two elastic bridge members. Each of the two opposing regions includes an opening for receiving a fastener. The fasteners may be any of those known in the art and may include screws, nails, pins, etc. In an unrestrained state, the two elastic bridge members are bowed outwardly. Prior to implantation, the bridge members can be reversibly strained inwardly so that the bridge members are nearly parallel (i.e., the bridge members are stretched laterally inwardly). A delivery device can hold the compression plate in the strained state prior to implantation. Upon implantation, the constraint on the bridge members is removed, and the bridge members attempt to return to their original unrestrained state, generating and maintaining a compressive load while healing occurs.

In another example, the ridged fixation device is a compression staple. The compression staple includes two elastic bridge members and two or more elastic legs. The bridge members and the legs meet at a pair of curved hinge regions, which are also elastic. In an unrestrained state, the legs of the staple are bent inwardly at an angle of less than 90°, and the bridge members are bowed outward. Prior to implantation, the bridge members can be reversibly strained inwardly so that the bridge members are nearly parallel (i.e., the bridge members are stretched laterally inwardly). The legs of the staple can be reversibly bent to a position perpendicular to a longitudinal axis of the bridge to allow for insertion of the staple into or across a prepared fracture site. A delivery device may be used to reversibly strain the bridge members and bend the legs to be parallel, to hold the compression staple in the strained state prior to implantation, and to insert the strained staple into or across the prepared fracture site. The constraint on the bridge members and the legs is removed, and the bridge members and the legs attempt to return to their original unrestrained state. This generates and maintains a greater (and more uniform) compressive load while healing occurs.

In another example, a rigid fixation device treats rib fractures. The rigid fixation device includes multiple compression staples that can be rigidly connected to a rod/thick piece of wire. One or more staples can be attached to one side of the fractured rib, and one or more staples can be attached to an opposite side of the fractured rib. A rod can be passed through the staples and rigidly locked to the staples. This creates a tension band between the two staples. During normal respiration, the rod will experience tensile forces, and the rib will experience compressive forces.

The compression staple includes a bridge and two elastic legs. The bridge and the legs meet at a pair of curved hinge regions, which are elastic. In an unrestrained state, the legs of the staple are bent inwardly at an angle of less than 90°. Prior to implantation, the legs of the staple can be reversibly bent to a position perpendicular to a longitudinal axis of the bridge to allow for insertion of the staple onto a rib. A delivery device may be used to reversibly bend the legs to be parallel, hold the staple in a strained state prior to implantation and to insert the strained staple into or across the prepared fracture site. The constraint on the legs is removed, and the bridge and legs attempt to return to an original unrestrained state, locking the staple onto the bridge. With the staple locked onto the rib, a rod can be passed through an opening on the bridge of the staple and rigidly locked to the staple, creating a rigid structure.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view detailing the principals of tension banding;

FIG. 2 is a schematic view of a compression plate system that can be used as a rigid fixation device for cardiothoracic surgery;

FIG. 3 is a schematic view of other compression plate shapes for cardiothoracic plating;

FIG. 4 is a schematic view of a compression staple that can be used as a rigid fixation device for cardiothoracic surgery; and

FIG. 5 is a schematic view of a rigid fixation system for the treatment of rib fractures.

### DETAILED DESCRIPTION OF THE PREFERRED EMBODIMENTS

There exists a clinical need for a rigid fixation device which generates and maintains compression between fractured bones.

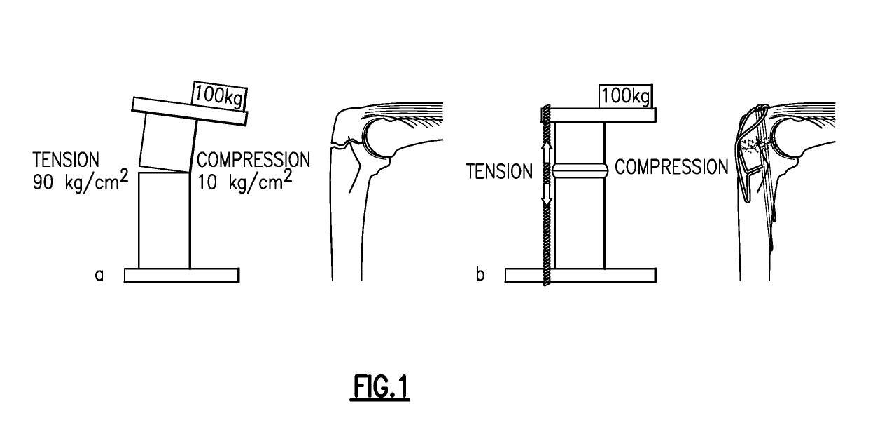

FIG. 1 shows a schematic detailing the principle of tension banding. When a bone is loaded with an eccentric load, the bone has a tension side and a compression side. A tension band converts a tensile force into a compression force at an opposite cortex. The tension band experiences tension, and the bone experiences compression. Compression aids in bone fracture healing per Wolf's Law.

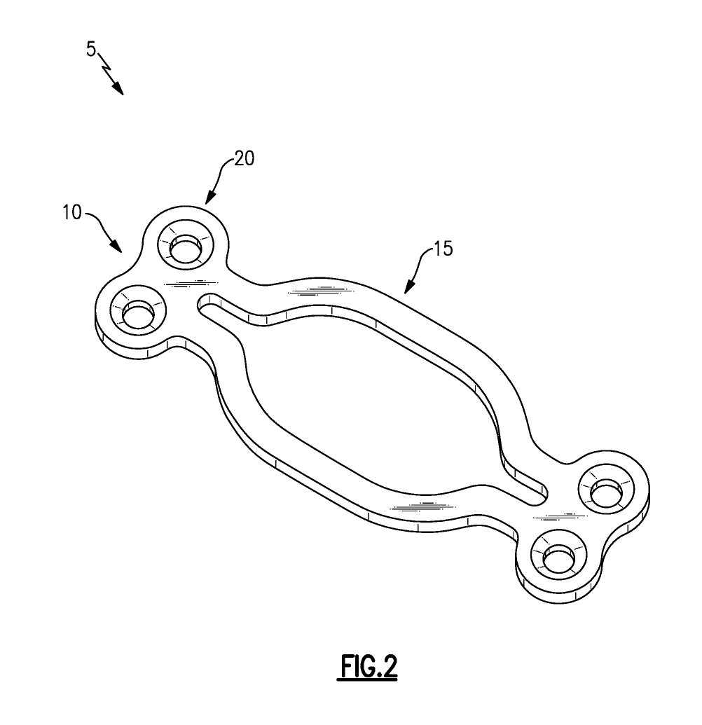

The principal of tension banding can be applied to a rigid fixation device used in cardiothoracic surgery. As shown in FIG. 2, a compression plate 5 is capable of turning tensile loads on an anterior surface of a sternum into compressive forces. Following a sternotomy, an anterior cortex will experience tensile forces (pulling the bones apart), and an opposite cortex will experience compressive forces (pushing the bones together). With a compression plate 5 attached to an anterior side of the sternum, the compression plate 5 will experience a tensile load and convert it to a compressive load that is imparted on a fracture.

The compression plate 5 is preferably a structure manufactured from a single piece of shape memory material (e.g., a material capable of exhibiting superelasticity and/or a temperature induced shape change). The shape memory material may be a metal alloy (e.g., Nitinol) or a polymer (e.g., appropriately processed PEEK). The compression plate 5 is designed to reduce fractures and generate and maintain compression between bone fragments to aid in healing.

The compression plate 5 generally includes two opposing regions 10 joined together by a pair of elastic bridge members 15. At least one opening 20 is formed in each of the two opposing regions 10 for receiving fixation screws. The openings 20 may have a countersunk feature (e.g., a bore-counterbore configuration) to allow heads of the fixation screws to sit substantially flat with a top surface of the compression plate 5. Additionally, the openings 20 may be threaded to allow for positive engagement between the openings 20 and the threaded fixation screws.

In an unrestrained state, the elastic bridge members 15 are bowed outwardly, such as in the manner shown in FIG. 1. Prior to implantation, the elastic bridge members 15 of the compression plate 5 can be reversibly strained inwardly (i.e., bent laterally inwardly), thus increasing the distance between opposing regions 10 (and therefore the openings 20) of the compression plate 5. For example, when the compression plate 5 is formed out of Nitinol, elastic deformations of up to approximately 8% are achievable. In one example, the nitinol is in an austenite or stress-induced martensite phase.

During implantation, the strained (i.e., elongated) compression plate 5 is positioned against and secured to the bone fragments by passing fixation screws through the openings 20 and into the bone fragments. Removal of the induced strain on the compression plate 5 (provided by a delivery device) results in the compression plate 5 attempting to return to the original unrestrained state, generating a compressive load on the bone (i.e., through the elastic bridge members 15, the openings 20 and the fixation screws extending through the openings 20) to maintain the compressive load on the bone during healing.

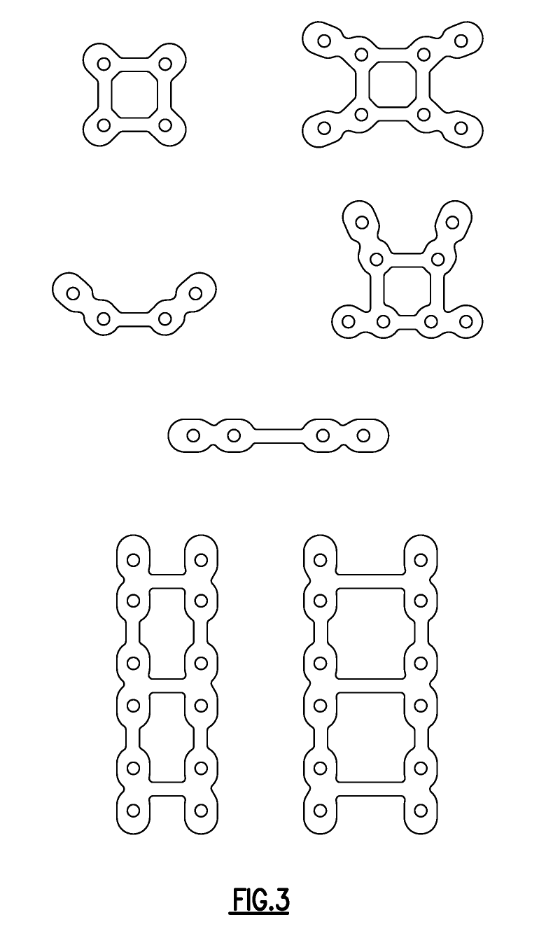

FIG. 3 shows other configurations and shapes of the compression plate 5.

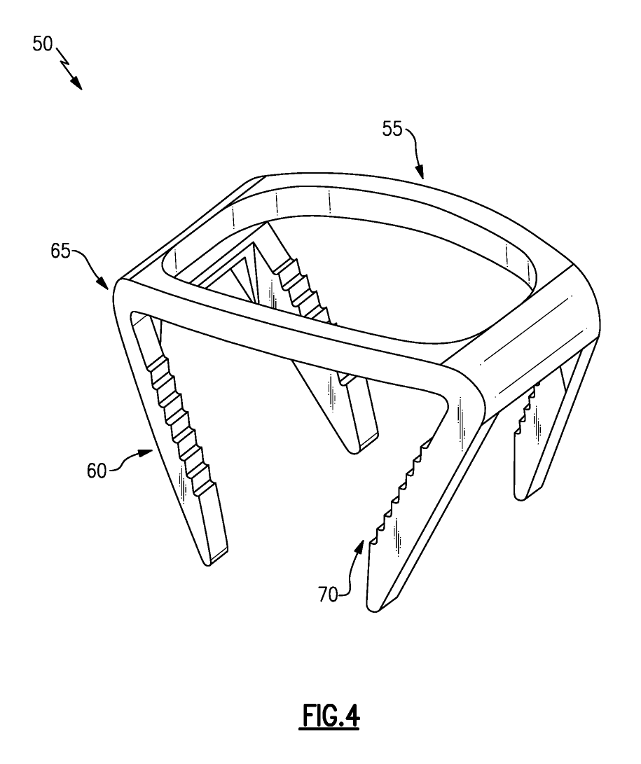

FIG. 4 shows a compression staple 50 capable of turning tensile forces into compression forces. The compression staple 50 is preferably formed of a single piece of shape memory material (e.g., a material capable of exhibiting superelasticity and/or a temperature-induced shape change). The shape memory material may be a metal alloy (e.g., Nitinol) or a polymer (e.g., appropriately processed PEEK). The compression staple 50 is designed to reduce fractures and generate and maintain compression between bone fragments to aid in fracture healing by converting tensile forces on an anterior surface to compressive forces.

The compression staple 50 includes two elastic bridge members 55 and two or more pairs of elastic legs 60. The bridge members 55 and the elastic legs 60 meet at a pair of curved hinge regions 65, which are elastic. The elastic legs 60 may include barbed teeth 70 to help the elastic legs 60 of the compression staple 50 grip into the bone after implantation and prevent the elastic legs 60 of the compression staple 50 from working their way back out of the bone.

In an unrestrained state, the elastic legs 60 of the compression staple 50 are bent inwardly with an angle of less than 90° defined between the elastic legs 50 and the bridge members 55. The bridge members 55 are bowed outward. In one example, two or more pairs of elastic legs 60 extend at an angle of about 45° relative to a longitudinal axis of the bridge members 55 when in the unrestrained state.

Prior to implantation, the bridge members 55 of the compression staple 50 can be reversibly strained inward (i.e., stretched longitudinally). The elastic legs 60 of the compression staple 50 can be reversibly bent to a position such that they are substantially perpendicular to the bridge members 55, allowing for insertion of the elastic legs 60 of the compression staple 50 into or across a prepared fracture site, with the stretched bridge members 55 of compression staple 50 spanning across the fracture line. For example, when the compression staple 50 is formed of Nitinol, elastic deformations of up to approximately 8% are achievable. In one example, the nitinol is in an austenite or stress-induced martensite phase.

A delivery device strains the bridge members 55 and bends the elastic legs 60, retaining the compression staple 50 in a strained state prior to implantation. The delivery device then inserts the compression staple 50 into or across a prepared fracture site. Upon insertion of the strained compression staple 50 into or across the prepared fracture site, the constraint on the bridge members 55 and the elastic legs 60 is removed, and the compression staple 50 attempts to return to the original unrestrained state. This movement generates a compressive load across the fracture line that is maintained during healing.

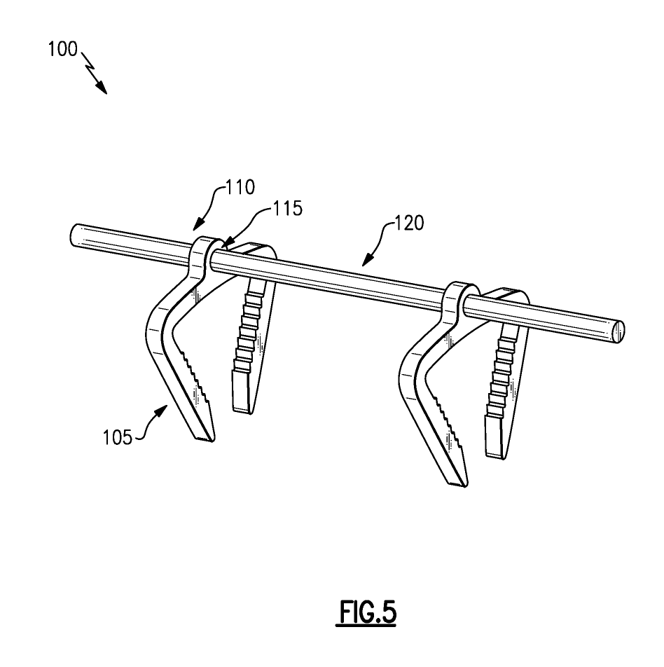

As shown in FIG. 5, a schematic of a rigid fixation system 100 for a rib fracture is shown. The rigid fixation system 100 includes two or more staples 105 implanted on a rib, with at least one of the staples 105 on each side of the fracture. The staples 105 includes a boss 110 including a hole 115. The hole 115 is sized to allow passage of a rod 120. The rod 120 can be rigidly fixed to the staples 105 by any means (e.g., interference fit, set screw). When the rod 120 is rigidly connected to the staples 105, a tension band is created that converts tensile forces on an anterior cortical surface to compression forces, stabilizing the rib fracture during healing. In one example, the nitinol is in an austenite or stress-induced martensite phase. In an embodiment, a compression device includes a first opposing region and a second opposing region. An elastic bridge member connects the first opposing region and the second opposing region. The elastic bridge member has a non-linear configuration when in an unbiased condition, and the elastic bridge member is capable of being elastically deformed to a more linear configuration upon an application of force to the elastic bridge member. A first opening is in the first opposing region and a second opening is in the second opposing region. The first opening and the second opening are each configured to receive a fastener. The first opening and the second opening are separated by a first distance when the elastic bridge member is in the non-linear configuration, and the first opening and the second opening are separated by a second distance when the at least one elastic bridge member is in the more linear configuration, and the second distance is greater than the first distance.

In another embodiment according to any of the previous embodiments, the compression device comprises a shape memory material.

In another embodiment according to any of the previous embodiments, the shape memory material includes Nitinol.

In another embodiment according to any of the previous embodiments, the Nitinol is in an austenite or stress-induced martensite phase.

In another embodiment according to any of the previous embodiments, the compression device is mounted to a delivery device that reconfigures the elastic bridge member from the non-linear configuration to the more linear configuration.

In another embodiment according to any of the previous embodiments, the fastener is disposed in each of the first opening and the second opening.

In another embodiment according to any of the previous embodiments, the fastener is a threaded fastening screw.

In another embodiment according to any of the previous embodiments, the compression device is a compression plate.

In another embodiment according to any of the previous embodiments, the compression device is a staple.

In another embodiment according to any of the previous embodiments, a method converts tensile forces to compressive forces at a cortical surface of a bone. The method includes the steps of applying a force to an elastic bridge member of a compression device. The bridge member is located between at least a first region and a second opposing region of the compression device. The elastic bridge member is elastically strained from a non-linear configuration when in an unbiased condition to a more linear configuration to position a first opening in the first region and a second opening in the second opposing region over a first bone fragment and a second bone fragment, respectively, with the elastic bridge member spanning a fracture line between the first bone fragment and the second bone fragment. The first opening and the second opening are separated by a first distance when the elastic bridge member is in the non-linear configuration, and the first opening and the second opening are separated by a second distance when the elastic bridge member is in the more linear configuration, and the second distance is greater than the first distance. The method includes the steps of passing a first fastener through the first opening and into the first bone fragment and passing a second fastener through the second opening and into the second bone fragment. Strain is released on the elastic bridge member so that the compression device applies a compressive force across the fracture line.

In another embodiment according to any of the previous embodiments, the compression device comprises a shape memory material.

In another embodiment according to any of the previous embodiments, the shape memory material includes Nitinol.

In another embodiment according to any of the previous embodiments, wherein the Nitinol is in an austenite or stress-induced martensite phase.

In another embodiment according to any of the previous embodiments, the method includes the step of mounting a delivery device to the compression device to reconfigure the elastic bridge member from the non-linear configuration to the more linear configuration.

In another embodiment according to any of the previous embodiments, the compression device is a compression plate.

In another embodiment according to any of the previous embodiments, the compression device is a staple.

In another embodiment according to any of the previous embodiments, a compression device includes a rod, a first staple, and a second staple. The first stable includes a first boss having a first hole and the second staple includes a second boss having a second hole, and the rod is received in the first hole and the second hole. Tensile forces in the rod are convertible to compressive forces across a fracture between a first bone fragment and a second bone fragment.

In another embodiment according to any of the previous embodiments, the compression device comprises a shape memory material.

In another embodiment according to any of the previous embodiments, the shape memory material comprises Nitinol.

In another embodiment according to any of the previous embodiments, wherein the Nitinol is in an austenite or stress-induced martensite phase.

Although a preferred embodiment of this invention has been disclosed, a worker of ordinary skill in this art would recognize that certain modifications would come within the scope of this invention. For that reason, the following claims should be studied to determine the true scope and content of this invention.

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A compression device comprising:
a first opposing region and a second opposing region;
an elastic bridge member that connects the first opposing region and the second opposing region, wherein the elastic bridge member has a non-linear configuration when in an unbiased condition, and the elastic bridge member is capable of being elastically deformed to a more linear configuration upon an application of force to the elastic bridge member; and
a first opening in the first opposing region and a second opening in the second opposing region, wherein the first opening and the second opening are each configured to receive a fastener;
wherein the first opening and the second opening are separated by a first distance when the elastic bridge member is in the non-linear configuration, and the first opening and the second opening are separated by a second distance when the at least one elastic bridge member is in the more linear configuration, and the second distance is greater than the first distance.

2. The compression device according to claim 1 wherein the compression device comprises a shape memory material.

3. The compression device according to claim 2 wherein the shape memory material comprises Nitinol.

4. The compression device according to claim 3 wherein the nitinol is in an austenite or stress-induced martensite phase.

5. The compression device according to claim 1 wherein the compression device is mounted to a delivery device that reconfigures the elastic bridge member from the non-linear configuration to the more linear configuration.

6. The compression device according to claim 1, wherein the fastener is disposed in each of the first opening and the second opening.

7. The compression device according to claim 6 wherein the fastener is a threaded fastening screw.

8. The compression device according to claim 1 wherein the compression device is a compression plate.

9. The compression device according to claim 1 wherein the compression device is a staple.

10. A method for converting tensile forces to compressive forces at a cortical surface of a bone, the method comprising the steps of:
applying a force to an elastic bridge member of a compression device, wherein the bridge member is located between at least a first region and a second opposing region of the device;
elastically straining the elastic bridge member from a non-linear configuration when in an unbiased condition to a more linear configuration to position a first opening in the first region and a second opening in the second opposing region over a first bone fragment and a second bone fragment, respectively, with the elastic bridge member spanning a fracture line between the first bone fragment and the second bone fragment, wherein the first opening and the second opening are separated by a first distance when the elastic bridge member is in the non-linear configuration, and the first opening and the second opening are separated by a second distance when the elastic bridge member is in the more linear configuration, and the second distance is greater than the first distance;
passing a first fastener through the first opening and into the first bone fragment and passing a second fastener through the second opening and into the second bone fragment; and
releasing strain on the elastic bridge member so that the device applies a compressive force across the fracture line.

11. The method according to claim 10 wherein the compression device comprises shape memory material.

12. The method according to claim 11 wherein the shape memory material comprises Nitinol.

13. The method according to claim 12 wherein the nitinol is in an austenite or stress-induced martensite phase.

14. The method according to claim 10 including the step of mounting a delivery device to the compression device to reconfigure the elastic bridge member from the non-linear configuration to the more linear configuration.

15. The method according to claim 10 wherein the compression device is a compression plate.

16. The method according to claim 10 wherein the compression device is a staple.

17. A compression device comprising:
a rod; and
a first staple and a second staple, wherein the first stable includes a first boss having a first hole and the second staple includes a second boss having a second hole, and the rod is received in the first hole and the second hole,
wherein tensile forces in the rod are convertible to compressive forces across a fracture between a first bone fragment and a second bone fragment.

18. The compression device according to claim 17 wherein the compression device comprises a shape memory material.

19. The compression device according to claim 18 wherein the shape memory material comprises Nitinol.

20. The compression device according to claim 20 wherein the compression device is formed of austenite or stress-induced martensite.
